(19) United States
(12) Patent Application Publication
(10) Pub. No.: US 2011/0182799 A1
(43) Pub. Date: Jul. 28, 2011

(54) SEQUESTRATION OF A GAS EMITTED BY AN INDUSTRIAL PLANT

(75) Inventors: Richard E. RIMAN, Belle Mead, NJ (US); Qinghua Li, Edison, NJ (US)

(73) Assignee: Rutgers, The State University of New Jersey, New Brunswick, NJ (US)

(21) Appl. No.: 13/008,464

(22) Filed: Jan. 18, 2011

Related U.S. Application Data
(60) Provisional application No. 61/297,646, filed on Jan. 22, 2010.

(51) Int. Cl. C01C 1/18; C01F 11/18; C01F 5/42; C01G 49/12; C01B 31/20; C07D 295/027; C07C 213/08; C07C 215/40

(52) U.S. Cl. 423/396; 423/431; 423/519.2; 423/561.1; 423/430; 423/438; 423/432; 544/358; 564/292

(57) ABSTRACT

A method of sequestering a multi-element gas emitted by an industrial plant is described herein, the method comprising: contacting a solution, including a first reactant comprising a multi-element gas emitted by an industrial plant and at least one gas absorber comprising nitrogen, for example ammonia or an amine, with a solid, including a second reactant, under conditions that promote a reaction between the first reactant and the second reactant to provide a first product, which incorporates one or more elements of the multi-element gas, thereby sequestering the multi-element gas.

This application claims priority to U.S. Provisional Application No. 61/297,646, filed on Jan. 22, 2010, which is incorporated herein by reference.

All references cited herein are incorporated by reference in their entirety. Cross-reference is made to U.S. application Ser. No. ______ (attorney docket number 32867.004.01) filed concurrently with this application.

### BACKGROUND

Gas emissions from industrial plants, like electrical power plants, such as coal-fired systems, are a major concern due to their immense volume—at tens of giga-ton (Gt) masses of carbon dioxide (CO₂) emission, which are emitted, on average, about 4 mega-tons (Mt)/year/plant (IPCC report, ISBN 92-9169-119-4). Currently, over 2100 coal-fired power plants (not to mention countless other industrial plants, including manufacturing plants, assembly plants and the like, which emit one or more gases) account for about 33% of all U.S. CO₂ emissions, corresponding to about 2 Gt/year (Science, vol. 317, 184 (2007)). Gas-fired and fuel oil-based plants are also a concern. In addition, industrial processes, such as steel making, glass melting and ceramic manufacturing, are large contributors to carbon dioxide emissions (e.g., cement, 0.79 Mt/year/plant). Steel making creates perhaps the highest emission among these processes, approaching that of coal-fired plants (3.5 Mt/y/plant). As mentioned above, many other manufacturing processes produce CO₂, such as aluminum and ammonia manufacturing.

Several conventional methods of carbon capture and sequestration (CCS) have been proposed and/or are used for emission control. One commonly employed method for post-combustion CCS involves the use of monoethanolamine (MEA). With this process, carbon dioxide in a flue gas forms an adduct with MEA, with the MEA-CO₂ adduct being soluble in an MEA or aqueous MEA solution. The adduct is subsequently sent to a stripping tower, where, at elevated temperatures, MEA is separated from the carbon dioxide. The MEA can be recycled or re-used to forming more MEA-CO₂ adduct. The carbon dioxide that was driven off from the adduct is then pressurized, transported and stored/injected underground either in geologic formations, oil wells, or retired mines. This method of forming an adduct, driving off carbon dioxide and subsequent underground storage of carbon dioxide, while qualifying as a method of sequestration, is capital intensive, expensive and hardly ideal, as discussed further below.

There are several drawbacks to the aforementioned process. First, the MEA method is only effective when the temperature is sufficiently low to facilitate carbon dioxide adduct formation and avoid MEA decomposition. Namely, the flue gas must first be cooled to about 50° C. or below prior to allowing the flue gas to come into contact with the MEA. Alternatively, the MEA solution must be cooled prior to exposure to the flue gas. Thus, there is an energy cost for cooling the solution resulting in parasitic power losses, which also generate CO₂ by virtue of the additional power demands placed on the power grid. Second, the stripping tower needed to separate carbon dioxide from MEA also has associated with it operational costs because of the significant amount of energy needed to heat the MEA-CO₂ adduct solution to about 120° C. The large volume of solution, which needs to be heated, leads to a large energy input and additional parasitic power loss to the power grid. Third, pressurization of the carbon dioxide step is also a high cost step with accompanying parasitic energy loss (by “parasitic” is meant that the energy produced by a power plant, which ideally would be transmitted via a gird to customers, is sapped by the energy needs of the conventional method being described), where compressors are needed to achieve supercritical pressures of about 14 MPa (2000 lb/in²) or more.

Although storage of the carbon dioxide by injection underground is possible for select locations around the world, the pipeline and maintenance costs further increase the capital investment and operational costs. Overall, these cost issues can increase the cost of products (e.g., electricity) by tens of a percent, with a number as high as 81% being reported.

Several approaches have been proposed to reduce the energy consumption. Addition of a small amount of primary or secondary amine to a tertiary amine is one method to enhance the absorption rate in the absorber while lowering the energy needs of the regeneration step in the carbon dioxide stripper. Nevertheless, a certain amount of energy consumption for the solvent regeneration is still unavoidable. In addition, the capitalization of conventional CCS equipment is costly, and thus the increase in costs for power plant construction can rise by as much as 87%. In addition to concerns related to cost, greenhouse gas emission, energy and electricity production rate, there are ecological concerns regarding the consequences of storing CO₂ in geological formations and beneath the ocean. Long-term storage of a gaseous substance is fraught with uncertainty and unknown hazards.

Thus, a need exists for a method that can be integrated with power generating, various manufacturing and other industrial activities to capture and sequester gases emitted by the plants in these industries, while at the same time reducing cost, parasitic energy loss, further greenhouse gas (GHG) and/or flue gas (FG) generation and minimizing the need for underground storage of the captured gases.

### SUMMARY OF THE INVENTION

A multi-faceted approach is provided toward reducing greenhouse and flue gas emissions from a wide range of manufacturing entities and activities. A mineral carbonation process is utilized to provide one of the embodiments in the multi-faceted approach. The methods described herein can have a negative or neutral carbon footprint and can separate and sequester GHGs and FGs emitted by an industrial plant, while at the same time produce useful byproducts, such as ceramics or compositions suitable for use in pharmaceuticals.

One embodiment provides a method of sequestering a multi-element gas, comprising: (i) providing a first solution comprising at least one gas absorber, the gas absorber comprises nitrogen and, in combination with a multi-element gas, is capable of forming an adduct; (ii) contacting the first solution with the multi-element gas to promote the formation of the adduct, the adduct comprising the at least one gas absorber and the multi-element gas, which adduct is then present in solution; (iii) providing a reactant comprising at least one element; (iv) allowing the first solution comprising the adduct to contact the reactant under conditions that promote a reaction, wherein the multi-element gas in the adduct reacts with the reactant to form at least a first product and a second product in the solution, wherein the first product comprises at least one element of the multi-element gas, and wherein the second product comprises at least one element of the at least one gas absorber and at least one element of the reactant.

The gas is thus separated from the gas absorber, which can be recycled for use in a subsequent gas absorption step or other process, thereby eliminating the need of a stripping tower in a power generating or other industrial plant.

In another embodiment, a method of forming an organic compound from of forming an organic compound from sequestering a multi-element gas comprising: (i) providing a first solution comprising at least one gas absorber, the gas absorber comprises nitrogen and, in combination with a multi-element gas, is capable of forming an adduct; (ii) contacting the first solution with the multi-element gas to promote the formation of the adduct, the adduct comprising the at least one gas absorber and the multi-element gas, which adduct is then present in solution; (iii) providing a multi-element reactant; (iv) allowing the first solution comprising the adduct to contact the reactant under conditions that promote a reaction, wherein the multi-element gas in the adduct reacts with the reactant to form at least a first product and a second product in the solution, wherein the first product comprises at least one element of the multi-element gas, and wherein the second product comprises at least one element of the at least one gas absorber and at least one element of the reactant; and (v) heating the solution in the presence of a catalyst to form a third product comprising at least one element of the second product, wherein the third product comprises an organic compound.

Another embodiment provides a ceramic produced by a greenhouse gas or flue gas sequestering process, which process comprises reacting at least one component of a porous matrix with an adduct comprising a greenhouse or a flue gas and at least one gas absorber comprising an amine, which adduct is carried by an infiltrating medium to contact the at least one component of the porous matrix to provide at least a first product, thereby producing a ceramic.

Another embodiment provides a nitrogen-containing compound produced by a gas separating or gas sequestering process, or a combination thereof, which process comprises reacting at least one component of a solid matrix with at least a first reactant that comprises at least one greenhouse or flue gas and which is present in a nitrogen containing infiltrating medium to provide at least a first product and a second product in the solution, wherein the first product comprises at least one element of the at least one greenhouse or flue gas, and wherein the second product comprises at least one element of the solid matrix and at least one element of the reactant; and heating the second product in the presence of a catalyst to form a third product comprising at least one element of the second product, whereby the third product produced comprises a nitrogen-containing compound.

### BRIEF DESCRIPTION OF THE DRAWINGS

FIG. 1 shows results of a thermodynamic simulation, illustrating the relationship between CaSiO₃ and varying NaOH concentration under ambient condition with 1 molal wollastonite. The reaction can be represented as 2CaSiO₃+4NaOH=Na₄SiO₄(s)+2Ca(OH)₂.

FIG. 2 shows results of a thermodynamic simulation, illustrating the relationship between Na₄SiO₄ and varying MEA concentration under ambient condition with 1 molal Na₄SiO₄. The reaction can be presented as MEA-CO₂(l)+Na₄SiO₄(s)+2Ca(OH)₂=2CaCO₃(s)+Na₄SiO₄(s)+MEA(l).

FIG. 3 shows results of a thermodynamic simulation, illustrating the relationship between anorthite and varying NaOH concentrations under ambient condition with 1 molal anorthite. The reaction can be presented as CaAl₂Si₂O₈+8NaOH=2Na₄SiO₄(s)+Ca(OH)₂+2Al(OH)₃.

FIG. 4 shows results of a thermodynamic simulation, illustrating the effect of Na₄SiO₄ and Al(OH)₃ solid on the reaction of MEA-CO₂, and Ca(OH)₂. Simulation is performed with 2 mol Na₄SiO₄(s)+1 mol Ca(OH)₂+2 mol Al(OH)₃+1 mol CO₂ with varying MEA concentrations under ambient condition. The reaction can be presented as MEA-CO₂(l)+2Na₄SiO₄(s)+Ca(OH)₂+2Al(OH)₃=CaCO₃(s)+MEA (l)+2Na₄SiO₄(s)+2Al(OH)₃.

FIG. 5 provides an X-ray diffraction pattern of CaCO₃ synthesized from Ca(OH)₂ pellets and MEA-CO₂ solution in one embodiment.

FIG. 6 provides an X-ray diffraction of CaCO₃ synthesized from CaSiO₃ pellets and MEA-CO₂ solution in another embodiment.

FIG. 7 provides an X-ray diffraction of CaCO₃ synthesized from CaCl₂ and MEA-CO₂ in yet another embodiment.

### DETAILED DESCRIPTION OF THE INVENTION

### Sequestration of Greenhouse Gas/Flue Gas

The gas being sequestered according to the methods as described herein can be any gas susceptible to sequestration. For example, the gas can be a greenhouse gas (GHG), a flue gas (FG), or a combination thereof. The source of these gases can be from an industrial plant, including, for example, a power generating plant or a manufacturing plant. A flue gas can be, for example, a gas emitted from an industrial plant, such as a product (or byproduct) of an industrial process. A greenhouse gas can be any gas that is commonly known as such, and can include a gas that comprises the element hydrogen, carbon, sulfur, phosphorus, oxygen, nitrogen, fluorine, or combinations thereof. The gas molecule can comprise one, two, three, or more elements (found in the periodic table). The elements may be the same or different. For example, a multi-element gas can be carbon dioxide, containing carbon and oxygen elements (atoms). Alternatively, the gas can be fluorine gas (i.e., F₂), comprising two fluorine atoms. The gas can also be a mixture of gas molecules. For example, the gas can include water vapor, carbon dioxide, methane, nitrous oxide, ozone, chlorofluorocarbons, hydrogen sulfide, sulfur oxide, sulfur trioxide, or combinations thereof. Alternatively, the multi-element gas can preferably comprise HF, SO₂, SO₃, CO₂, F₂, H₂S, or combinations thereof. The terms greenhouse gas and flue gas herein are used interchangeably regardless of the source of the gas.

Sequestration of a gas generally refers to storing a gas in a permanent fashion, such as, for example, fixing the gas permanently in a solid or liquid phase, preferably in a solid phase. However, the term “sequestration,” as employed herein, can encompass a more general process that includes at least one of capturing, separating, and storing a gas. A capturing process can be a process that absorbs or “captures” an emitted gas before the gas is permanently stored. Similarly, a gas “sequesteror” can refer to a material that captures, separates, and/or stores a gas molecule.

### General Conditions for Gas Sequestration

The sequestration method described herein can be any suitable method that captures and/or permanently stores a gas in a solid or liquid phase. One such method can be hydrothermal liquid phase sintering (HLPS). HLPS can be used to make monolithic dense bodies. Such process can also be integrated into an industrial plant, such as a power plant, to sequester the GHG or FG generated and/or emitted by the plant. A description of the HLPS process can be found in, for example, U.S. application Ser. No. 12/271,566 (US Pub. No. 2009/0143211) and Ser. No. 12/271,513 (US Pub. No. 2009/0142578) to Riman et al., the disclosures of which each of which is incorporated in its entirety by reference herein.

In a preferred embodiment of HLPS, a “green” or partially sintered, porous, solid matrix having contiguous interstitial pores can be transformed into a sintered ceramic by the action of a liquid phase infiltrating medium. HLPS can be carried out under relatively mild conditions, frequently not exceeding the temperature and pressure encountered in a functioning autoclave. HLPS can be performed in a wide range of temperatures and pressures. For example, in some embodiments, the HLPS conditions can include temperatures less than about 2000° C., such as less than about 1000° C., such as less than about 500° C., such as less than about 200° C., such as less than about 100° C., such as less than about 40° C., such as room temperature. The reaction gauge pressure can be less than about 100000 psi, such as less than 70000 psi, such as less than about 50000 psi, such as less than about 10000 psi, such as less than about 5000 psi, such as less than about 2000 psi, such as less than about 1000 psi, such as less than about 500 psi, such as less than about 100 psi, such as less than about 50 psi, such as less than about 10 psi. In one embodiment, the hydrothermal sintering process can be carried out at a temperature in the range of about 80° C. to about 180° C. and at a pressure in the range of about 1 to about 3 atmospheres. (1 atmosphere is about 15 psi.) Note that in this embodiment because the pressure refers to gauge pressure, the real pressure can be calculated by adding the gauge pressure to 1 atmosphere. Any starting matrix material that is capable of undergoing a hydrothermal reaction with an infiltrating species to produce a different substance may be used to produce the hydrothermally sintered product. Hence, a wide variety of starting materials may be selected, depending on the contemplated end use. The materials can be formed into a solid matrix having the desired shape and size and, subsequently, subjected to the steps of the method described herein for transformation into the sintered finished product. The solid matrix can be a porous solid matrix or a substantially dense solid matrix. The matrix is described in more detail below.

“Hydrothermal reaction” described herein can include transformations taking place in aqueous or nonaqueous liquid media. Furthermore, such transformations may include the dissolution and re-precipitation of the same chemical species, the dissolution of one chemical species and its combination with a second chemical species to form a composite material in which the initial chemical species remain distinct, or the reaction of one chemical species with a second chemical species to produce a new chemical moiety that is distinct from the starting species. The hydrothermal sintering process thus can fill the interstitial spaces or voids in a porous solid matrix with a moiety by precipitation (or re-precipitation), ion addition, ion substitution, or a combination thereof. The moiety can comprise the same chemical species as that in the solid matrix, a composite resulting from the co-re-precipitation of two distinct chemical species, a new product resulting from a reaction between two chemical species, a re-precipitated material derived from an infiltrant species contained in the medium, or combinations thereof.

In one embodiment, an HLPS process can be carried out under conditions in which at least a portion of the mass of the green porous solid matrix reacts with preselected infiltrant species present in the fluid medium to produce a new product. For example, in one embodiment of the invention, the infiltrant species may be the gas of the gas-amine adduct described above, the fluid medium may be the solution in which the gas-amine adduct is dissolved.

The shape of the product can be retained from that of the solid matrix. In one embodiment, when the molar volume of the product is greater than that of the oxide powder (i.e., a positive molar volume change—i.e., transformation to a larger molar volume), the nucleated product fills the voids of the compact and increases its density. The molar volume change need not be positive; it can also be negative (i.e., transformation to a smaller molar volume) or no change depending on the ion species and reaction mechanism. For example, a portion of the matrix can dissolve away during the reaction, increasing porosity while creating new chemical bonding and a negative molar volume change. Similarly, if the new material form has the same volume as that from the loss of the matrix, then there is substantially no molar volume change.

HLPS reaction can occur via, for example, ion addition, and/or ion substitution. Ion addition reactions take place when ions (anions or cations) in the infiltrating medium can be added to the matrix host without substituting another ion in the matrix. Examples of an ion addition can include transformation from oxide to hydroxide, or from oxide to carbonate. Examples of an ion substitution can include transformation from hydroxide to carbonate, or hydroxide to oxalate. Additionally, the reaction can occur via disproportionation, wherein the insoluble inorganic host/matrix material can be split into two insoluble inorganic products. Disproportionation can be performed, for example, for oxides, fluorides, hydroxides, sulfates, mixed metal oxides, silicates, hydroxyapatites.

Heterogeneous nucleation can also take place during the reaction. As described previously, the change in density can depend on the type of the matrix material and/or that of the product formed. Once the hydrothermal reaction is completed, the open pores can be further removed by, for example, aging. After the reactions as described above are completed, the densified monolithic matrix may be rinsed or bathed in a solution to wash away excess infiltrating solution. The rinsing solution can be any suitable solution, such as, for example, ammonium acetate at pH 5. In one embodiment, the densified matrix may be subsequently dried in an oven at a temperature of about room temperature to about 300° C., such as about at 90 to 250° C. The residual porosity that may be present in the sintered ceramic can be further removed by heating to a higher temperature, such as between 500° C. and 700° C., such as about 600° C.

The product sintered by the HLPS process can be in the form of a ceramic. Such a ceramic can have a variety of applications. For example, it can be used as a structural, chemical (e.g., catalyst, filtration), electronic component, semiconductor material, electrical material, or combinations thereof. Alternatively, the product produced can be a composition that has certain properties that allow it to be used as a (part of a) pharmaceutical composition or that comprises such a composition.

### Gas Sequestration

The HLPS-based reaction process can occur via a dissolution-re-precipitation reaction mechanism. Alternatively, the reaction can occur via an ion-substitution reaction. In the former, small portions of the compacted porous solid matrix can dissolve, furnishing dissolved species which can react with the ions in the infiltrant solution; the ions in the infiltrant solution can be metal ions. In one embodiment, the amount of the infiltrant added can be enough to produce the complete reaction in a single step. Alternatively, multiple steps can be involved. For example, multiple infiltrations can be involved. In one embodiment, strontium titanate can be formed from a titania matrix, thereafter by another infiltration it can form strontium apatite. Alternatively, via multiple infiltrations, a carbonate can be formed, which can then form a protective oxalate layer. In another embodiment, the compact can be partially infiltrated and dried, and the infiltration step can be repeated until the final product is produced.

An example of using an HLPS-based reaction to sequester a GHG or FG can involve contacting and subsequently reacting at least two reactants. The first reactant can be in a solution and can be in the form of an “infiltrating species” or “infiltrant” referring to at least a gas and at least a gas absorber. The gas-absorber can be a nitrogen-based absorber. For example, it can comprise ammonia or an amine such as monoethanolamine (MEA), diethanolamine (DEA), methyldiethanolamine (MDEA), 2-amino-2-methyl-1-propanol (AMP), or combinations thereof. The gas absorber is described in detail in a later section. The gas can be any multi-element GHG or FG. The gas absorber can comprise an amine and can further form an adduct with the gas. The adduct can be formed by, for example, absorption of the gas by the gas absorber. The second reactant can be a part of a matrix and can be, for example, a multi-element reactant. A separate later section describes the matrix in further detail. In this embodiment, the first and second reactants can react under a controlled environment for a plurality of products. For example, a first product can incorporate one or more elements of the gas. As a result of transforming the gas into at least one reaction product, the gas is thus sequestered.

In one embodiment of a gas sequestration method, the gas absorber can form an adduct with the gas as the gas is emitted by the industrial plant. Namely, a solution comprising at least one amine-based gas absorber is allowed to contact a gas that is emitted by an industrial plant. The gas absorber can be any suitable absorber that is capable of absorbing a gas. In this embodiment, the absorption of the gas by the gas absorber thus can produce a reactant, such as a first reactant described above, which reactant can further react with a matrix. Alternatively, the first reactant need not be formed while the gas is being emitted. For example, the gas sequestration described herein can be applied to a sequestration process wherein the gas was supplied to be the processed “pre-absorbed.” In other words, the first reactant is supplied to the sequestration herein “as is,” rather than being formed during the sequestration process. One example of such a pre-formed first reactant can be that the first reactant is formed at a location different from the location in which the sequestration is to take place.

Various types of products can be formed as a result of the methods described herein. For example, the products can be a multi-element composition, in which one of the elements comes from a gas emitted from a plant. For example, the composition can comprise (or is) a pharmaceutical composition or a ceramic. In one embodiment, the product can comprise an inorganic compound, including, for example, a ceramic, as described above. Alternatively, it can be, an organic compound, such as a nitrogen-containing organic compound. Such an organic compound can be further processed to form at least one further compound. Alternatively, the reaction can create a plurality of product compounds. For example, in one embodiment, the reaction between the adduct and the reactant present in a solid matrix forms a first product. This product can comprise at least one element of the gas—i.e., the element “C” if the gas is carbon dioxide (CO₂). In this embodiment, a second product can be created, and it can comprise at least one element of the reagent from the infiltrating solution/medium and at least one element of the reactant from the matrix.

In one embodiment, a nitrogen-containing compound is produced as a result of the sequestration process. In this embodiment, a solid matrix is reacted with a solution comprising at least a reagent, which can comprise an adduct further comprising at least one GHG or FG and a gas absorber. The reagent can be in an infiltrating medium. The reaction can be as described above. As a result of the reaction, a first product is formed, comprising at least one element of the gas. Also formed can be a second product comprising at least one element of the solid matrix and at least one element of a gas absorber. Additional steps can be applied. For example, an additional step can be applied to increase the concentration of any of the products. For example, in one embodiment, the concentration of the second product is increased as a result of a heating step to further form a third product that comprises at least one element of the second product. The third product can be, for example, a pharmaceutical composition or a portion thereof. For example, the pharmaceutical composition can be piperazine. The third product can also comprise monoethanolammonium nitrite. Alternatively, it can comprise ethanamide. In another embodiment, the third product can comprise a precursor to a different product. In one embodiment, the precursor is a precursor to a nitrous oxide gas (i.e., laughing gas). In this embodiment of a nitrous oxide gas, a catalyst need not be used during the heating step.

The heating step can be carried out, for example, in the presence of a catalyst. The catalyst can comprise, for example, a halide salt of a metal. The metal can be, for example, zinc, iron, aluminum, magnesium, or mixtures thereof. The catalyst can be a dehydration catalyst, such as the halide salts of zinc, iron, aluminum or magnesium, or mixtures thereof. The heating step can be carried out in any suitable temperature, depending on the materials involved. For example, it can be from about 100° C. and 500° C., such as about 150° C. to about 400° C., such as about 200° C. to about 300° C., such as 220° C. to about 250° C.

Depending on the materials, any of the products described can be inorganic or organic, and the products among themselves need not be the same. For example, the first product can be organic, but the second product can be inorganic, and vice versa. The inorganic product can be, for example, a ceramic. For example, the ceramic can comprise a carbonate. In one embodiment, the first product can comprise sulfide, sulfite, sulfate, carbonate, or combinations thereof. In one carbon separating/sequestering embodiment, a gas absorber, such as carbon gas absorber, first forms an adduct in a solution with a GHG or FG. The adduct subsequently reacts with a solid matrix, such as porous solid matrix having interstitial spaces. The infiltrating solution comprising the adduct fills a portion of the interstitial spaces to promote a reaction between the adduct and a reactant present in the matrix to form a product, such as a ceramic product. The product can contain at least one element of the gas.

Alternatively, the product can be an organic product, such as a nitrogen-containing organic compound. Such a compound can be for example, one that is suitable for use in a pharmaceutical composition, such as piperazine. Alternatively, the compound can comprise a precursor to nitrous oxide, or monoethanolammonium nitrate.

After the product(s) is formed, the gas absorbed can be further processed to be released from the adduct. As a result, the gas absorber can be recycled and reused in a next sequestration or other process.

Any of the products described above can be further processed. For example, in an embodiment wherein ceramic granules (as green compacts) are formed, the granules can flow via standard conveying systems to automated dry presses. These presses are capable of stamping out shapes at rates as high as thousands of parts per minute. Thus, mass production of monolithic ceramic green bodies can be possible. These green bodies can be subsequently subjected to HLPS for densification, thus made strong for a wide range of applications, such as structural materials for construction applications. Accordingly, in one embodiment, one byproduct of the GHG or FG sequestration can be ceramic. The product can also be used as proppants, aggregate for roofing or landfill applications. In one embodiment, the use of granules has advantages, including substantially minimizing the need for a spray drying unit, thus substantially lowering the costs for energy, labor, and raw materials.

### Matrix

The matrix can be a variety of types of solids. In one embodiment, it is desirable to have a matrix that can react with the infiltrating species or adduct, which will be described further below, to form a solid product. The product from the reaction can be insoluble, and it is desirable in one embodiment, that no liquid (such as water) is formed during the reaction. It is noted that upon additional processes, such as a further reactive chemistry process, the product can be used as a reactant to form further products, which then can be a solid or a liquid. Such further process is described below.

In one embodiment wherein the matrix is a solid matrix, the matrix, or alternatively, the starting material, can be in the form of a powder compact. A solid matrix can be in the form of a dense solid or a porous solid. For example, the porosity of the solid matrix can be of any desirable value, such as over about 20%, such as over about 40%, such as over about 60%, such as over about 70%, such as over about 80%, such as over about 90%. In one embodiment, a granulated powder can be used as a feedstock, which can be pressed into a shaped object. Many techniques of processing for the granulated powders, including spray drying, can be used, although some of these techniques can be costly. In one embodiment, the heat of the flue gas in the power plant can be used as a drying medium to form ceramic granules for fabricating the compact. The powder compact can also be part of a slurry. In one embodiment, a ceramic slurry can be sprayed into a scrubbing tower. The slurry can comprise one or more powders and the powders can comprise a wide range of materials, such as ceramic. For example, the slurry can comprise a carbonate, such as calcium carbonate, which can be used to scrub GHG or FG, such as those comprising sulfur, including SO₂. The gas scrubbing can be performed before or after the spray drying. In one embodiment, the spray-drying tower can be part of a post-process to GHG or FG scrubbing.

The slurry can be sprayed by any method that is generally known in the art, including, for example, by co-current or counter-current flows. Any scrubber suitable for a plant can be used. For example, scrubbers such as the commercial ones designed by Babcock and Wilcox (B&W) can be suitable for making and collecting the ceramic granules.

The matrix can comprise a reactant that contacts and/or reacts with another reactant in the infiltrating medium, as described above. The reactant in the matrix can further comprise at least one of a gas separator and a gas sequesteror.

In one embodiment, a porous solid matrix is derived from an oxide powder, such as a metal oxide powder and/or a ceramic. The powder may be amorphous or crystalline, preferably crystalline. Moreover, the metal oxide powder may have a wide range of particulate sizes ranging from a mean particle size of about 0.01 micron to about 100 microns, including, for example, about 0.02 to about 50 microns, such as about 0.04 to about 20 microns, such as about 0.08 to about 10 microns. In one embodiment, the powder has a mean particle size ranging from about 0.1 micron to about 5 microns.

The metal in the metal oxide can be chosen from an oxide of a Group IIa metal, Group IIb metal, Group Mb metal, Group IVb metal, Group Vb metal, transition metal, lanthanide metal, actinide metal, or mixtures thereof. Preferably, the chosen metal oxide or the sintered finished product can have potential chemical, ceramic, magnetic, electronic, superconducting, mechanical, structural or even biological applications. The sintered finished product can have industrial or household utility. The finished product need not necessarily comprise the same material as the reactants. For example, a product substantially free of barium titanate, BaTiO₃, may be produced by reactants that comprise barium and/or titanium. However, in a different embodiment, the barium and/or titanium comprising reactant (or reactants) can act mostly as an intermediate reaction species, and thus may not necessarily be included in the final product.

The matrix can comprise at least one reactant that will react with the infiltrating species from an infiltrating medium/solution, as described below. The reactant can comprise at least one element, such as one, two, or three elements. The matrix can comprise at least one industrial waste. For example, the matrix can comprise red mud, coal, gypsum, wood, or garbage in general generated by a plant. The matrix can be a byproduct from a process carried out in a plant or can be one prepared specifically for the purpose of gas sequestration. For example, in one embodiment, the matrix is a solid matrix that is generated from a hot flue gas generated by the plant.

In the case of a solid matrix, the solid matrix can comprise a material that does not dissolve in a solution readily. In one embodiment, the porous solid matrix is derived from powder. The powder can be of any kind. For example, it can be a metal oxide powder. Examples of suitable a metal oxide can include the oxides of beryllium (e.g., BeO), magnesium (e.g., MgO), calcium (e.g., CaO, CaO₂), strontium (e.g., SrO), barium (e.g., BaO), scandium (e.g., Sc₂O₃), titanium (e.g., TiO, TiO2, Ti₂O₃), aluminum (e.g., Al₂O₃), vanadium (e.g., VO, V₂O₃, VO₂, V₂O₅), chromium (e.g., CrO, Cr2O₃, CrO3, CrO₂), manganese (e.g., MnO, Mn₂O₃, MnO₂, Mn₂O₇), iron (e.g., FeO, Fe₂O₃), cobalt (e.g., CoO, CO₂O₃, CO₃O₄), nickel (e.g., NiO, Ni₂O₃), copper (e.g., CuO, Cu₂O), zinc (e.g., ZnO), gallium (e.g., Ga₂O₃, Ga2O), germanium (e.g., GeO, GeO₂), tin (e.g., SnO, SnO₂), antimony (e.g., Sb₂O₃, Sb₂O₅), indium (e.g., In₂O₃), cadmium (e.g., CdO), silver (e.g., Ag₂O), bismuth (e.g., Bi₂O₃, Bi₂O₅, Bi₂O₄, Bi₂O₃, BiO), gold (e.g., Au₂O₃, Au₂O), zinc (e.g., ZnO), lead (e.g., PbO, PbO₂, Pb₃O₄, Pb₂O₃, Pb₂O), rhodium (e.g., RhO₂, Rh₂O₃), yttrium (e.g., Y₂O₃), ruthenium (e.g., RuO₂, RuO₄), technetium (e.g., Ti₂O, Ti₂O₃), molybdenum (e.g., MoO₂, Mo₂O₅, Mo₂O₃, MoO₃), neodymium (e.g., Nd₂O₃), zirconium (e.g., ZrO₂), lanthanum (e.g., La₂O3), hafnium (e.g., HfO₂), tantalum (e.g., TaO₂, Ta₂O5), tungsten (e.g., WO₂, W2O₅), rhenium (e.g., ReO₂, Re₂O₃), osmium (e.g., PdO, PdO₂), iridium (e.g., IrO₂, IR₂O₃), platinum (e.g., PtO, PtO₂, PtO₃, Pt₂O₃, Pt₃O₄), mercury (e.g., HgO, Hg₂O), thallium (e.g., TiO₂, Ti₂O₃), palladium (e.g., PdO, PdO₂) the lanthanide series oxides, the actinide series and the like. Moreover, depending upon the particular application involved, mixtures of metal oxides may also be used in making the preform.

The matrix can also comprise a hydroxide, such as a metal hydroxide. For example, it can comprise magnesium hydroxide (e.g., Mg(OH)₂), calcium hydroxide (e.g., Ca(OH)₂), strontium hydroxide (e.g., Sr(OH)₂), and barium hydroxide (e.g., Ba(OH)₂), chromium hydroxide (e.g., Cr(OH)₂), titanium hydroxide (e.g., Ti(OH)₂), zirconium hydroxide (e.g., Zr(OH)₂), manganese hydroxide (e.g., Mn(OH)₂), iron hydroxide (e.g., Fe(OH)₂), copper hydroxide (e.g., Cu(OH)₂), zinc hydroxide (e.g., Zn(OH)₂), aluminum hydroxide (e.g., Al(OH)₃), or a combination thereof.

The matrix can also comprise a fluoride, such as a metal fluoride. For example, it can comprise magnesium fluoride (e.g., MgF₂), calcium fluoride (e.g., CaF₂), strontium fluoride (e.g., SrF₂), and barium fluoride (e.g., BaF₂), chromium fluoride (e.g., CrF₂), titanium fluoride (e.g., TiF₃), zirconium fluoride (e.g., ZrF₄), manganese fluoride (e.g., MnF₂), iron fluoride (e.g., FeF₂), copper fluoride (e.g., CuF₂), nickel fluoride (e.g., NiF₂), zinc fluoride (e.g., ZnF₂), aluminum fluoride (e.g., AlF₃), or combinations thereof.

The matrix can also comprise a mixed metal oxide, such as a metal titanate. For example, it can comprise magnesium titanate (e.g., MgTiO₃), calcium titanate (e.g., CaTiO₃), strontium titanate (e.g., SrTiO₃), barium titanate (e.g., BaTiO₃), or combinations thereof.

The matrix can also comprise a sulfate, such as a metal sulfate. For example, it can comprise magnesium sulfate (e.g., MgSO₄), calcium sulfate (e.g., CaSO₄), strontium sulfate (e.g., SrSO₄), barium sulfate (e.g., BaSO₄), chromium sulfate (e.g., Cr2(SO₄)₃), titanium sulfate (e.g., TiSO4, Ti₂(SO₄)₃), zirconium sulfate (e.g., ZrSO₄), manganese sulfate (e.g., MnSO4), iron sulfate (e.g., FeSO4), copper sulfate (e.g., CuSO₄), nickel sulfate (e.g., NiSO₄), zinc sulfate (e.g., ZnSO₄), aluminum sulfate (e.g., Al₂(SO₄)₃), or combinations thereof.

The matrix can also comprise a silicate or a hydrated silicate, such as a metal silicate or a metal hydrated silicates. For example, it can comprise lithium metasilicate, lithium orthosilicate, sodium metasilicate, beryllium silicate, calcium silicate, strontium orthosilicate, barium metasilicate, zirconium silicate, manganese metasilicate, iron silicate, cobalt orthosilicate, zinc orthosilicate, cadmium metasilicate, andalusite, silimanite, hyanite, kaolinite, magnesium silicate, magnesium hydrated silicate, calcium hydrated silicates, or combinations thereof. The matrix can also comprise minerals, such as mineral silicates, such as inosilicate (wollastonite), such as neosilicates (olivine), such as phyllosilicate (serpentine), such as tectosilicate (feldspar), or combinations thereof.

The matrix can also comprise an aluminosilicate, such as a metal aluminosilicate. For example, it can comprise calcium aluminosilicate, sodium calcium aluminosilicate, potassium calcium aluminosilicate, sodium magnesium aluminosilicate, or combinations thereof.

The matrix can also comprise a hydroxyapatite, such as a metal hydroxyapatite. For example, it can comprise calcium carbonate, calcium nitrate tetrahydrate, calcium hydroxide, or combinations thereof.

The matrix can further comprise an inert fill material, in addition to any of the materials mentioned above and others. An inert fill material can be any material that is incorporated into the solid matrix to fill the pores and does not significantly react with the infiltrating species for chemical bonding. For example, the inert material can be wood, plastic, glass, metal, ceramic, ash, or combinations thereof.

In the case of powder, the powder can be characterized by a mean particle size, which can range from about 0.005 μm to 500 μm, such as from about 0.01 μm to about 100 μm, by particle size distribution and by specific surface area. A fine mean particle size and a narrow particle size distribution can be desirable for enhanced dissolution. The powder can be formed into a green body of any desired shape and size via any conventional technique, including extrusion, injection molding, die pressing, isostatic pressing, and slip casting. Ceramic thin films can also be formed. Any lubricants, and/or binders of similar materials used in shaping the compact can be used and should have no deleterious effect on the resulting materials. Such materials are preferably of the type that evaporate or burn out on heating at relatively low temperatures, preferably below 500° C., leaving no significant residue.

The matrix can comprise, for example, a mineral, industrial waste, or an industrial chemical material. A mineral can be, for example, a mineral silicate, iron ore, periclase, or gypsum. Industrial waste can be, for example, iron (II) hydroxide, fly ash, bottom ash, slag, glass, oil shells, red mud, battery waste, recycled concrete, mine tailings, paper ash, or salts from concentrated reverse osmosis brine. An industrial chemical can be any chemical synthesized or prepared by a factory or an industry in general.

The compact can be formed into the shape and the dimensions of a product material of a predetermined shape and size. The compact can be in any form. The volume of open porosity of the compact (0-80%) can depend on the ratio of molar volume of the reaction product to the molar volume of the powder. The product material can be, for example, a monolithic body, such as a monolithic dense body. In one embodiment, the reaction product formed within the pores of the compact can have a greater molar volume than the powder. The reaction product can have a greater molar volume than the oxide powder to fill the voids of the compact during the reaction. For example, if the molar volume of the reaction product is twice as large as that of the oxide powder, the compact should have an open porosity of about 50% by volume.

The pores in the starting powder compact can be small, for example, between about 0.01 micrometers (μm) and about 100 μm, such as between about 0.1 μm and about 1 μm, and uniformly distributed throughout the compact, thereby enabling the infiltrant solution to fully penetrate the powder compact. The pore volume content, both close and open porosity, and the pore size can be determined by standard methods. For example, a mercury intrusion pore sizer can be used to evaluate these three parameters.

The reactant material for the matrix can be any of those described previously. Further, the reactant in the matrix can be formed from a precursor of a reactant. For example, the reactant can be formed by reacting a reactant precursor with another agent, such as a basic solution. The basic solution can comprise, for example a hydroxide, such as a metal hydroxide. In one embodiment, the solid matrix can be prepared at least in part by the heat that is generated by the hot gas, such as a FG, generated by an industrial plant.

### Infiltrating Medium

As described previously, hydrothermal sequestration methods can make use of aqueous or non-aqueous media. The choice of liquid solvent can depend on the infiltrant species that may be a part of the infiltrating medium. The term “infiltrating species” herein generally refers to any molecule that is in the infiltrating medium solution. The species can have a substantial solubility in the liquid solvent under the conditions of the hydrothermal sintering process. For example, if the infiltrant species are ionic, then a liquid solvent can be water. Certain nonionic infiltrants may also possess sufficient solubility in aqueous media. Alternatively, the species can be a reactant comprising a gas adduct, which can comprise a gas absorber and a gas molecule that is absorbed by the gas absorber. In the case of being used to sequester a greenhouse or flue gas, the medium is interchangeably referred to as “gas capture solution.” The infiltrating species can act as a reactant that reacts with another reactant in the matrix. In one embodiment, the species can comprise a gas absorber and a gas molecule. The gas molecule can be absorbed by the gas absorber. The gas absorber can have very minimal chemical reaction with the gas molecule during absorption.

The gas absorber can be any type of molecule that absorbs gas. For example, it can be an amine-based gas absorber. The amine can be any type of amine known in the art. The amines can, for example, comprise a primary amine, a secondary amine, a tertiary amine, a quaternary or combinations thereof. It can also comprise ammonia; alkanolamines; polyamines of a mixed or single type; cyclic and aromatic amines; aminoacids; and sterically free and hindered amines. The amine can also comprise monoethanolamine (MEA), diethanolamine (DEA), ethyldiethanol amine, methyldiethanolamine (MDEA), 2-amino-2-methyl-1-propanol (AMP), 3-piperidino-1.2-propanediol, 3-quinuclidinol, 2-piperidineethanol, 2-piperidinemethanol, N,N-dimethylethanolamine, 2-amino-2-methyl-1,3 propanediol, diisopropanolamine, piperazine, or combinations thereof. Additional materials can also be found in Puxty et al., Environ. Sci. Technol., 2009, 43, 6327-6433. Alternatively, the amine can be a proprietary amine, such as Flexsorb, KS-1, KS-2, KS-3, or combinations thereof; a description of these amines can be found in, for example, Gupta et al., CO2 capture technologies and opportunities in Canada. 1^{st} Canadian CC&S technology roadmap workshop, 18-19 Sep. 2003, Calgary, Alberta, Canada.

For instance, the gas absorber can comprise ammonia, monoethanolamine (MEA), diethanolamine (DEA), methyldiethanolamine (MDEA), 2-amino-2-methyl-1-propanol (AMP), or combinations thereof. In certain instances, surfactants (e.g., polysiloxanes, polyethylene glycols, and alkyldimethylamine oxides and the like) may be added to the infiltrating medium.

The gas absorber can further comprise a base, such as a basic solution, such as an organic base and/or an inorganic base. An organic base can be any organic material can for example be an amine-based gas absorber, such as MEA. It can also be a polymer that acts as a Lewis base. For example, an organic base can be a pyridine. The inorganic gas absorber can comprise a material comprising a halide element. The infiltrating medium preferably contains water-soluble salts, such as metal salts (i.e., metal in ion forms). The cation of such salts, for example, may come from the following metals: beryllium, magnesium, calcium, strontium, barium, scandium, titanium, vanadium, chromium, manganese, iron, cobalt, nickel, copper zinc, aluminum, gallium, germanium, tin, antimony, indium, cadmium, silver, lead, rhodium, ruthenium, technetium, molybdenum, neodymium, zirconium, ytterbium, lanthanum hafnium, tantalum, tungsten, rhenium, osmium, iridium, platinum, gold, mercury, thallium, palladium, cations of the lanthanide series metals, cations of the actinide series metals, or a mixture thereof. Alternatively, the cation can be ammonium.

In general, the anion of the salts dissolved in the infiltrating solution may come, for example, from the following groups: hydroxides, nitrates, chlorides, acetates, formates, propionates, phenylacetates, benzoates, hydroxybenzoates, aminobenzoates, methoxybenzoates, nitrobenzoates, sulfates, fluorides, bromides, iodides, carbonates, oxalate, phosphate, citrate, and silicates, or combinations thereof. The molar ratio of the metal ions contained in the infiltrant to the metal ion of the oxide powder can be selected to achieve a desired stoichiometric reaction product. Excess metal ions in solution may be needed to help achieve completion of the reaction.

Depending on the infiltrating medium and the matrix material, the resultant sintered product can be, for example, a titanate, if a material comprising titanium is involved. For example, titanates having an ilmenite structure can be obtained from TiO₂ and salts of Fe²⁺, Mg²⁺, Mn²⁺, Co²⁺, Ni²⁺, or a combination thereof, in water. Titanates having the perovskite structure can be prepared from aqueous salt solutions of Ca²⁺, Sr²⁺, barium ions, or a combination thereof. Moreover, compounds having a spinel structure can be obtained, including Mg₂TiO₄, Zn₂TiO₄, and CO₂TiO₄. Furthermore, different phases of barium titanate, such as that having the formula BaₓTi_{y}O_{x+2y}, in which x and y are integers, can be obtained by the method of the present invention.

Alternatively, the resultant sintered product can be a carbonate, sulfate, oxalate, or combinations thereof; materials that can be used can include a material that may decompose before it is able to sinter if a conventional sintering method is used. For example a carbonate will decompose into its oxide when heated before it is able to sinter in a conventional sintering method. The carbonate, sulfate, or oxalate, can be, for example, metal carbonate, metal sulfate, or metaloxalate, respectively, comprising a cation of an element found on the periodic table.

### Industrial Plants

The HLPS-based sequestration processes described here can integrate with existing industrial plants to provide a compact, energy efficient, and environmentally friendly process for sequestering the GHG and/or FG generated by the plants. The process can be versatile, using a wide range of raw materials, which will allow processes to be used for ingredients that can be conveniently accessible (e.g., waste from a factory), thereby minimizing transportation costs.

One such a plant can be an industrial plant that emits a gas, such as a multi-element gas. The plant can comprise one or more apparatuses by which a gas emitted by the industrial plant is sequestered. By an apparatus, a reaction is promoted between a first reactant, which is comprised of the gas and at least one gas absorber comprising nitrogen, for example an amine, and a second reactant, which is provided by a solid, to produce at least one product. The product can incorporate one or more elements of the multi-element gas, thereby sequestering the multi-element gas.

The GHG or FG, such as carbon dioxide, can come from a variety of sources. For example, in one embodiment, carbon dioxide is captured from the flue gas (“post-combustion capture”) at a low temperature, such as less than about 200° C., such as less than about 100° C., such as at about 50° C., such as at about room temperature. The carbon sequestering process described herein can be integrated with other available industrial practices, such as industrial separation, pre-combustion, and oxyfuel-based approaches. In one embodiment, the process can capture more than 70%, such as more than 80%, such as more than 90%, such as more than 95%, of the GHG or FG produced.

The plant can be any type of industrial plant, such as a manufacturing plant, a power-generating plant, such as an electrical power generating plant, a processing plant, or a combination thereof. The plant can be one that utilizes a solid fuel, a liquid fuel, a gaseous fuel, or combinations thereof. The solid fuel can comprise coal, the liquid fuel can comprise fossil fuel, and the gaseous fuel can comprise natural gas (e.g., methane, ethane, octane, etc). Because of the sequestration methods described herein do not need to involve a stripping tower, the plant need not have a stripping tower. A stripping tower can be used to separate a gas from a mixture comprising the gas, wherein the mixture can comprise a gas absorber. Alternatively, the plant can have a stripping tower, but the tower is not actively involved in the sequestration process described herein.

The industrial plants described herein can have a gas separation or stripping tower or have no stripping tower. In either case, the method described herein does not involve using the stripping tower to separate the gas from the conventionally used MEA, thereby saving a tremendous amount of energy (and cost). The gases can be any type of gas, such as a GHG or FG, including carbon dioxide, or a gas containing, in general, carbon, sulfur, phosphor, nitrogen, hydrogen, oxygen, or combinations thereof. The plant can be any type of industrial plant, such as an electrical power plant. Alternatively, it can also be a plant involved in industrial processes, including cement, fertilizer, metal (e.g., steel, aluminum), or glass. A plant can utilize any type of fuel, such as a solid fuel, a liquid fuel, a gaseous fuel, or combinations thereof. The solid fuel can be, for example, coal; the liquid fuel can be, for example, fossil fuel; and the gaseous fuel can be, for example, a natural gas.

### Characterization of the Products

HLPS can produce a sintered product with a very homogeneous and very fine microstructure. The porosity of the sintered material can be, for example, less than about 15 percent, such as less than about 10 percent, such as less than about 5 percent, or even practically be fully dense. The total porosity of the compact can be determined by a standard technique, for example, with a mercury pore sizer. Density can be estimated using a conventional technique such as Archimedes' mercury pore sizer.

One characteristic of the sintered material undergoing the HLPS process is that it can have the same shape, or even size, as the starting green compact. In one embodiment wherein the product undergoes substantially no molar volume change, no shrinkage of the compact may result, which is in contrast to many ceramic manufacturing processes, and thus little or no machining of the sintered material is needed.

Composition of Sintered Material

As illustrated in the Examples, a broad range of chemical compositions can be used to make the sintered material. Furthermore, the number of different metal oxides and salts involved in the formation of the sintered material need not be restricted in any particular way. In addition, the stoichiometry of the final product can be dictated by the molar ratios of reactants present in the green compact and infiltrating medium. The composition of the sintered material can be evaluated using Quantitative X Ray Diffraction (QXRD) and Inductively Coupled Plasma (ICP).

Microstructure and Related Mechanical Properties

The product of the HLPS process can have a microstructure that substantially resembles a net-like interconnecting network. The monoliths obtained from the HLPS process can also exhibit composite structures such as a core-shell structure. In addition, the product can have superior mechanical properties, such as high tensile strength, compressive strength, and desirable tensile modulus. This strengthening can arise from the chemical bonding formed during the process between the physically bonded particles by ion substitution, ion addition, Ostwald ripening (i.e., recrystallization that can form new network), or combinations thereof. In one embodiment, Ostwald ripening can involve aging a carbonate material in an alkaline medium. Furthermore, in the case where there is a positive molar volume change, densification can be achieved, as described previously.

Instead of making ceramics using high temperature processes, HLPS can provide an alternative method to form a variety of ceramic materials of interest in a fluid in mild temperature and/or pressure conditions. The ceramic can be of any type. For example, it can comprise crystalline inorganic materials, amorphous inorganic materials, conventional ceramics, or combinations thereof. The crystalline inorganic materials can comprise, for example, nonmetals, such as carbon nitride, metallic and nonmetallic species, such as quartz, titanium nitride, yttria, calcium fluoride, or a combination thereof. The amorphous inorganic materials can comprise, for example, nonmetals, such as amorphous carbon, or metallic and nonmetallic species, such as silica, zirconium-lanthanum, aluminum-sodium-fluoride, silicon oxynitride, or combinations thereof.

The ceramic crystals can be made without using “corrective” milling processes as in a conventional procedure and can have crystal sizes and morphology with suitable reactivity for making materials. Furthermore, the chemical bonds of the product produced by HLPS can be ceramic bonds substantially free of hydraulic bonds. Conventional cements have hydraulic bonding, and as a result, can degrade with respect of their mechanical strength, starting at about 200° C.—they can lose almost all of their strength at 1000° C. By substantially minimizing formation of hydraulic bonding (that is, bonding involving water molecules or portions thereof), the ceramic produced by HLPS can withstand temperatures of at least about 1000° C.

The hydraulic-free bonds may include various different types of bonds that may be found in ceramics. For example non-hydraulic bonds may include hydrogen bonds, such as a between fluoride ion and a hydroxyl group, or between a phosphate group and a hydroxyl group. Non-hydraulic bonds may also include ionic bonds, covalent bonds, bonds that have partially ionic and covalent character, or bonds that have a covalent and/or metallic character such as for example the bonding found in ceramics like titanium diboride, TiB2.

Other benefits of using HLPS to produce cement, or ceramic in general, can include shorter reaction time to form a ceramic product. Hydrothermal reactions can be based on aqueous solution reactions where ceramics can be directly formed from a solution at temperatures typically less than about 400° C., such as less than 300° C., or at about room temperature.

The ceramic produced can also be highly dense with minimal consumption of water and substantially no hydraulic bonds and mostly ceramic bonds. For example, the bonds in cements can be created by hydration of the powders slurred in water. Conventional ceramics have most of their bonds created by diffusion induced by high temperature firing. By contrast, the ceramics of HLPS can be formed by reacting a monolithic compact of powder or solid matrix with an infiltrating medium to fill the interstitial spaces (i.e., pores) of the particles. The crystals nucleating and growing in these interstitial spaces can form chemical bonds to one another and to the powder matrix to create a ceramic-bonded monolithic body. As a result, unlike a hydraulic cementation processes, anhydrous ceramic bonds can be formed, whose stability can be at least about 1000° C., such as about 2000° C. Furthermore, unlike a conventional densification process (e.g., solid state sintering), the reaction temperature can be lower than about 90° C., such as 50° C., such as room temperature.

As described previously, a product of HLPS process can undergo a change in molar volume (increase or decrease) or substantially no change. In one embodiment, wherein the molar volume change is positive, densification can also occur. In one embodiment, wherein the solid matrix can act as a scaffold for a bonded structure to form, substantially no change in dimensions has occurred. As a result, substantially no defects, such as cracks or defects, can be induced. While the material does not change dimension, the relative porosity of the structure can be controlled by the choice of the reactive chemistry where the percentage molar volume change between the product and reactant can determine the porosity remaining in the structure. For example, a 50% porous structure that is reacted to form a product that has a 100% molar volume change can fully densify. In one embodiment, having a large pore size can be desirable to achieve complete conversion. Note that the initial density can be controlled by both choice of the matrix powder and the forming technique for packing the powders.

There are many reactions where a volume increase or decrease can be engineered to result in a change of porosity while bonding the ceramic with the crystals that form from the reaction. For example, converting a matrix of CaSO₄ to CaC₂O₄.H₂O can result in a molar volume increase (densification) of 44.4 vol %, while converting CaSO₄ to CaCO₃ can result in a molar volume decrease (increased porosity) of −19.7 vol %. Control of this process can be further controlled by mixing components of negative and positive volume changes to engineer a composite whose net density (pore fraction) change can be engineered to either a zero, positive or negative value. In one embodiment, a molar volume increase of as large as 616 vol % and a molar volume decrease of 50.2 vol % can be possible.

The ability to decrease or increase porosity can have great utility. For example, large molar volume increases can have utility in low density matrices that can accommodate the large expansion, such as aggregates that can go into road building material or building structures. On the other hand, large volume decreases can be used to improve transport or reacting solutions that bond aggregates as the reactions proceed by increasing the permeability as the reaction proceeds. In addition, composites can include the addition of inert powders to reduce a density increase (or decrease), which can proportionately diminish the molar volume increase (or decrease). In general, whether the reaction results in a volume expansion or contraction of the matrix, crystals forming from the reaction can serve to bond the matrix, be it a reactant, inert component, or a product that has already formed from the reaction.

HLPS can provide a method to form ceramic bonds with a variety of materials, including those that cannot be prepared by any conventional process. Accordingly, ceramic such as marble, a naturally occurring calcium carbonate (CaCO₃), can be synthesized from non-carbonated calcium sources.

Because of the versatility of the HLPS process, the process can be used to capture GHG or FG's, such as carbon dioxide, while forming a dense ceramic, as previously described. The process can be further integrated into power-generating facilities that emit GHG or FG, wherein the gases can be captured and fed directly into the HLPS process as a reactant.

### Use of MEA

With the HLPS process, gases can be captured (and subsequently stored) from the atmosphere or directly from the emissions of an industrial plant, such as a power plant, and used in the reaction to form a variety of ceramics, including marble or cements, as products. The gas capture solution can contain a high concentration of the GHG or FG's. In one embodiment, the highly concentrated solution of CO₂ is complexed with a gas absorber, such as a carbon absorber, such as an amine, or such as MEA to form an adduct. MEA, as described previously, can be used as a reagent in carbon-capture applications. The solution that contains MEA can also contain other chemicals, such as ammonia. With a suitable solid matrix, a thermochemical driving force can then be employed to dissociate the amine from the CO₂ such that the gas can be free to react with the solid matrix.

MEA can be subsequently dissociated from CO₂ via several methods. For example, a reaction of the gas can take place directly with a solid oxide to form a product, while dissociating the MEA. For example, CO₂ can react directly with an oxide such as FeO to form a carbonate and free MEA, as follows: FeO+CO₂=FeCO₃.

Therefore, a GHG or FG, such as carbon dioxide, can be extracted from a gas-amine adduct (e.g., carbon dioxide-MEA adduct) to produce dissociated (“free”) MEA and a sintered ceramic monolith. The free MEA can be recycled to be used in a subsequent gas-capture application, and the entire process does not have a need for an MEA stripping tower. In addition to substantially minimizing the need for a stripping tower, the HLPS process that is used to capture GHG or FG emitted by a plant also substantially minimizes the need to pressurize the sequestered gasses to be stored underground, as the product is thermodynamically stable. Furthermore, by using the flue gas to process the granules, cooling of the flue gas prior to gas capture is not needed.

Other amine-based gas absorbers, which are described elsewhere herein, can be used in a fashion similar to that described herein for MEA to obtain comparable results.

### Precipitation of Metal Carbonates

In one alternative embodiment, CO₂-adsorbed MEA aqueous solution (MEA-CO₂) can react with an alkaline-earth metal hydroxide, such as Ca(OH)₂ or Mg(OH)₂, to produce carbonates and regenerate MEA with a low energy demand and a fast reaction rate. The first process could be described as follows:

Step 1): Formation of Alkaline Earth Metal Hydroxide from the Caustic Aqueous Solution

2MOH+M′Mₓ″O_{y}(s)=M′(OH)₂(s)+M₂Mₓ″O_{y}(l), (1-1)

2MOH+M′Mₓ″O_{y}(s)=M′(OH)₂(s)+M₂Mₓ″O_{y}(s), (1-2)

wherein, MOH is a caustic solid or solution (e.g., NaOH, KOH or any waste caustic solid/solution from the industry); M′Mₓ″O_{y} can be alkaline earth metal-oxide bearing materials, such as sulfate, silicate, and aluminosilicate minerals or industrial waste product (e.g., gypsum (CaSO₄), wollastonite (CaSiO₃), olivine (Mg₂SiO₄), anorthite (CaAl₂Si₂O₈), or combinations thereof). The product of M₂Mₓ″O_{y} can be either soluble or insoluble in the water.

### Step 2): Formation of Alkaline Earth Metal Carbonates and Regeneration of Mea

MEA-CO2(l)+2M′(OH)2(s)=M′CO3(s)+MEA(l) (2-1)

MEA-CO2(l)+2M′(OH)2(s)+M2Mx″Oy(s)=M′CO3(s)+MEA(l)+M2Mx″Oy(s) (2-2)

If the produced M₂Mₓ″O_{y} is dissolved in the solution (reaction (1-1)), an additional separation process can be used to recover M′(OH)₂ solid, which is used for the carbonation step (see reaction (2-1)). If the produced M₂Mₓ″O_{y} is a solid (see reaction (1-2)), this solid likely would not affect the reaction of MEA-CO₂ and M′(OH)₂. At the end of the reaction, only MEA solution is liquid phase, along with the carbonate solid and M₂Mₓ″O_{y} solid (see reaction (2-2)).

Both carbonation reaction (2-1) and (2-2) can be instantaneous reactions under ambient condition with mild agitation. Thus, fixation of CO₂ and regeneration of amine can be time and energy efficient processes.

### NON-LIMITING WORKING EXAMPLES

### Example A

### CO₂ Sequestration and MEA Regeneration from Mineral (Alumino)Silicates

### Example A1

### Wollastonite (CaSiO₃)

2CaSiO₃+4NaOH→Na₄SiO₄(s)+2Ca(OH)₂ Reaction 1

MEA(aqueous solution)+CO₂→MEA-CO₂(l) Reaction 2

MEA-CO₂(l)+Na₄SiO₄(s)+2Ca(OH)₂→2CaCO₃(s)+Na₄SiO₄(s)+MEA(l) Reaction 3

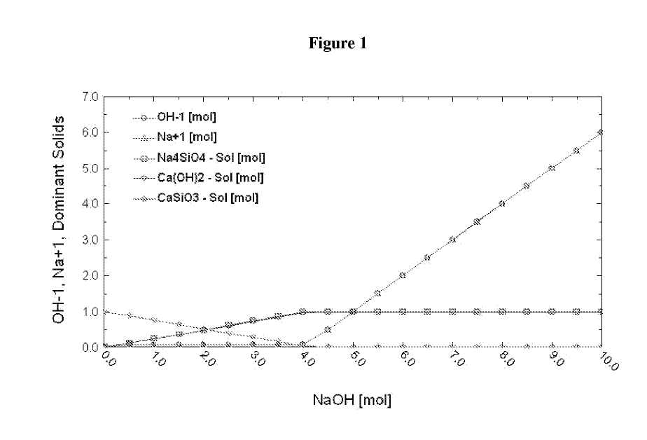

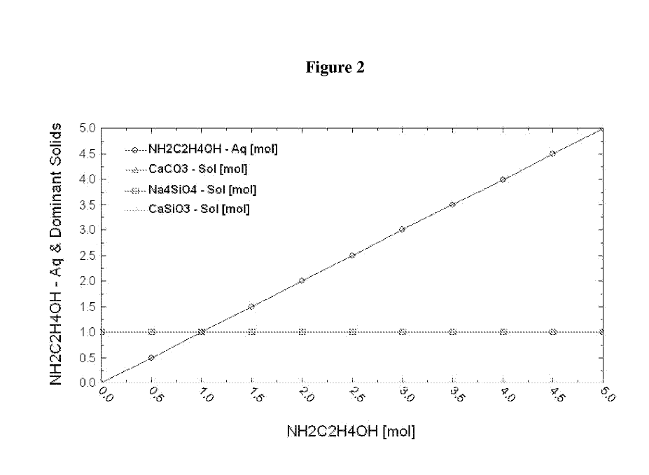

Thermodynamic simulation shows that when [NaOH] is about 4 M, 1 molal wollastonite is completely dissolved (as shown by x-intercept of the curve representing wollastonite in FIG. 1) to produce 1 mol Ca(OH)₂ and 1 mol Na₄SiO₄ solid (see FIG. 1) and Na₄SiO₄ solid has no influence on the reaction of MEA-CO₂ and Ca(OH)₂ (see FIG. 2).

1) 2 g CaSiO₃ was added into 100 ml 4M NaOH solution and heated for 6 hours under 500 rpm agitation at 90° C. 7 g Ca(OH)₂ and 9 g Na₄SiO₄ were formed. CO₂ saturated 30 wt % MEA solution was added into the solid formed as described above and the solution was stirred at 500 rpm for 10 minutes. XRD analysis of the final product suggests the presence of CaCO₃.

### Example A2

### Anorthite (CaAl₂Si₂O₈)

CaAl₂Si₂O₈+8NaOH→2Na₄SiO₄(s)+Ca(OH)₂+2Al(OH)₃ Reaction 1

MEA(aqueous solution)+CO₂→MEA-CO₂(l) Reaction 2

MEA-CO₂(l)+2Na₄SiO₄(s)+Ca(OH)₂+2Al(OH)₃→MEA(l)+CaCO₃(s)+2Al(OH)₃(s)+2Na₄SiO₄(s) Reaction 3

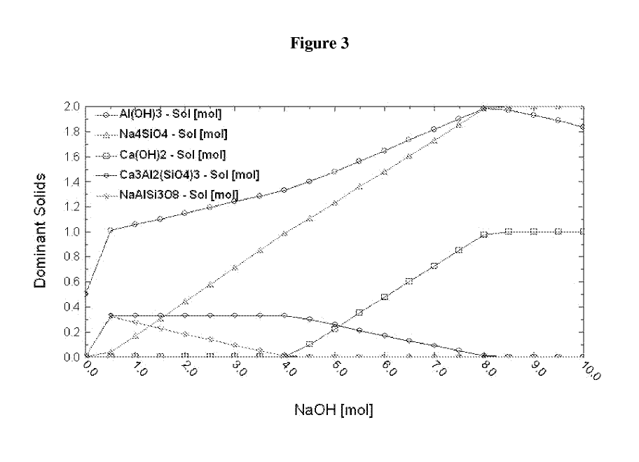

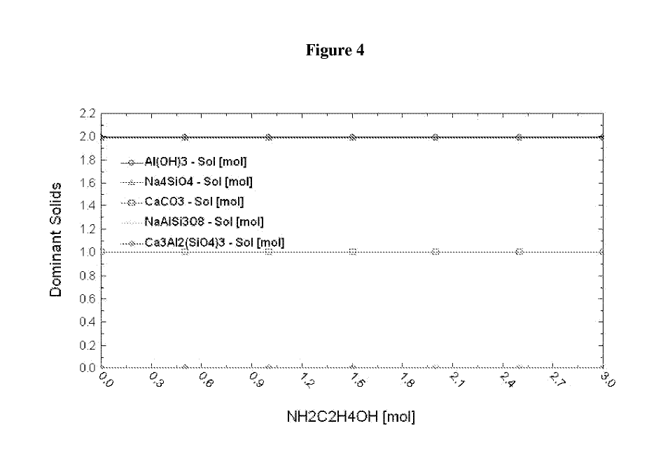

Thermodynamic simulation shows that when [NaOH] is about 8 M, 1 molal anorthite can be completely dissolved to produce 1 mol Ca(OH)₂ and 2 mol Na₄SiO₄ solid and 2 mol Al(OH)₃ (see FIG. 3). Al(OH)₃ and Na₄SiO₄ solids have no influence on the reaction of MEA-CO₂ and Ca(OH)₂ (see FIG. 4).

28 g CaAl₂Si₂O₈ was added into 100 ml 8 M NaOH solution. The solution was heated at 90° C. for 1 day under 500 rpm agitation. 7 g Ca(OH)₂, 36 g Na₄SiO₄ and 15 g Al(OH)₃ were produced. CO₂-saturated 30 wt % MEA solution was added into the solid formed as described above and the solution was stirred at 500 rpm for 10 minutes. XRD analysis of the final product suggested the presence of CaCO₃.

### Example B

### HLPS Carbonates and MEA Regeneration

### Example B1

### HLPS Ca(OH)₂ Pellet

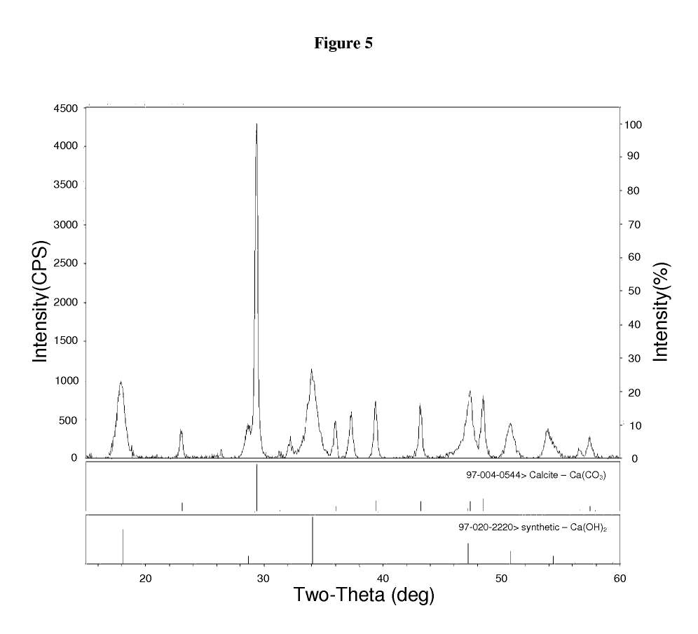

About 10 g Ca(OH)₂ was mixed with 10 g de-ionized water. The slurry was then shaken and poured into a 1″ diameter stainless steel die. A load of 6 tons was applied on the die slowly. The pressed Ca(OH)₂ pellets were dried at 95° C. for 1 day. The dried pellets were placed in the CO₂-saturated 20 wt % MEA solution at room temperature for 1 day. After 1 day reaction, the pellets were dried at 60° C. for 4 hours, then 95° C. for overnight. The dried pellets were placed into the CO₂-saturated 20 wt % MEA solution at room temperature for another 1 day reaction. The pellets were then taken out and thoroughly rinsed with de-ionized water. The pellet was dried in the oven at 60° C. for 4 hours, then 95° C. overnight. XRD analysis revealed the pellet comprised >50% CaCO₃ (calcite) (see FIG. 5). The sample maintained its shape and size and did not change its dimensions after HLPS. The material was mechanically stable.

### Example B2

### HLPS Wollastonite (CaSiO₃) Pellets

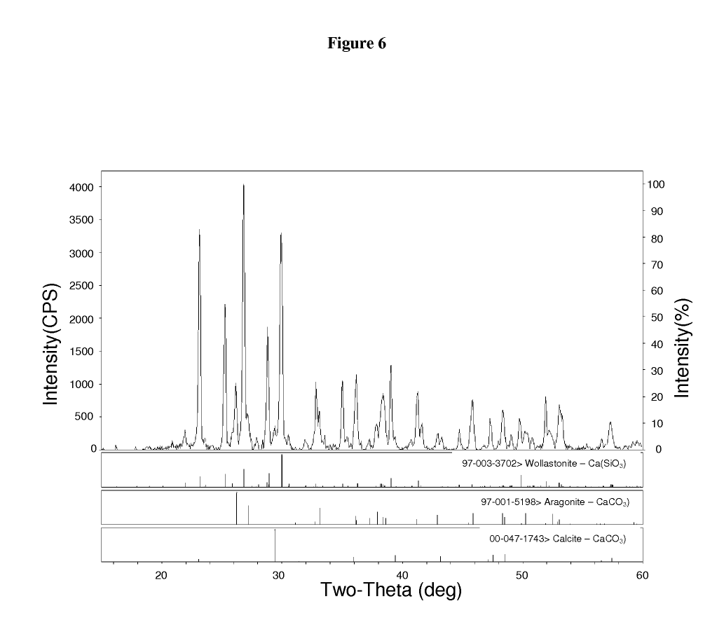

About 35 g CaSiO₃ was mixed with 35 g de-ionized water. The slurry was then shaken and poured into a 1″ diameter stainless steel die. A load of 6 tons was applied on the die slowly. The pressed CaSiO₃ pellets were placed in the CO₂-saturated 30 wt % MEA solution at 60° C. for 3 days. After 3 day reaction, the pellets were dried in the oven at 95° C. for overnight. The dried pellets were then placed in the CO₂-saturated 30 wt % MEA solution at 60° C. for another 2 day reaction. After 2 days, the pellets were dried as described above, and then placed in the CO₂-saturated 30 wt % MEA solution at 60° C. for 2 days. Subsequently, the pellets were dried as described above. The total reaction time was 7 days. XRD analysis revealed the pellet comprised CaCO₃ patterns (calcite and aragonite polymorphs) (see FIG. 6). The sample maintained its shape and size and did not change its dimensions after HLPS. The material was mechanically stable.

### Example C

### Sequestration of GHGs and Formation of Other Chemicals

### Example C1

### Sequestration of CO₂ and Formation of Piperazine

CaCl₂(s)+CO₂—(OH—C₂H₄—NH₂)(l)→CaCO₃(s)+OH—C₂H₄—NH₃₊.Cl₋(l) Reaction 1

MEA(aqueous solution)+CO₂→MEA-CO₂(l) Reaction 2

Reaction 3:

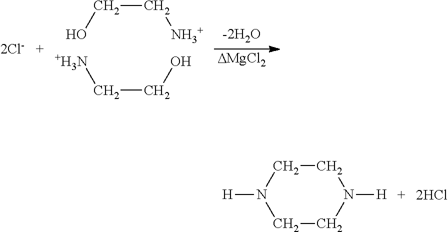

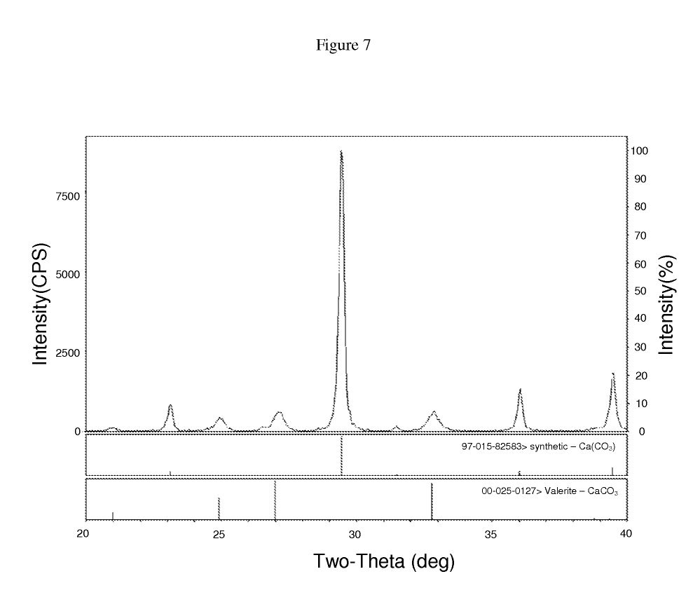

20 g CaCl₂ solid was added into 88 g CO₂-saturated 30 wt % MEA solution. After stiffing at 500 rpm for 5 minutes, the reaction was stopped. The solid product was separated by the filtration. XRD result indicated that the solid product was pure calcium carbonate (see FIG. 7). The filtrate was heated under 220-250° C. in the presence of dehydrated magnesium chloride catalysts. Hydrogen chloride was dissociated and about 18 g piperazine was formed.

### Example C2

### Sequestration of SO₂ and Formation of Piperazine

MEA(aqueous solution)+SO₂→MEA-SO₃(l) Reaction 1

MgCl₂+MEA-SO₃→MgSO₃(s)+MEA-Cl Reaction 2

Reaction 3:

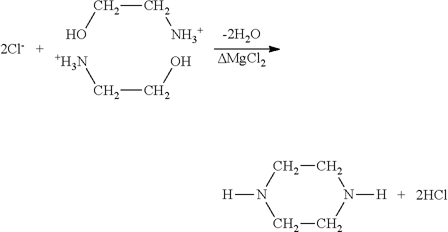

24 g MgCl₂ was added into 146 g SO₂-saturated 30 wt % MEA aqueous solution (about 6 g SO₂ was absorbed). After stirring at 500 rpm for 5 minutes, the reaction was stopped. The solution was separated by filtration. The solid was magnesium sulfite. The filtrate was heated under 220-250° C. in the presence of dehydrated magnesium chloride catalysts. Hydrogen chloride was dissociated and piperazine was formed.

### Example C3

### Sequestration of H₂₅ and Formation of Piperazine

MEA(aqueous solution)+H₂S→MEA-S(l)+H₂O Reaction 1

FeCl₂+MEA-S→FeS(s)+MEA-Cl Reaction 2

Reaction 3:

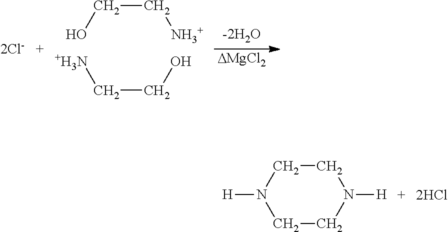

32 g FeCl₂ was added into 139 g H₂S-saturated 30 wt % MEA aqueous solution (about 8.5 g SO₂ was absorbed). After stirring at 500 rpm for 5 minutes, the reaction was stopped. The solid was separated by filtration. The solid was FeS. The filtrate was heated under 220-250° C. in the presence of dehydrated magnesium chloride catalysts. Hydrogen chloride was dissociated and piperazine was formed.

### Example C4

### Sequestration of CO₂ and Formation of Laughing Gas

NH₄OH+CO₂→(NH₄)₂CO₃(l) Reaction 1

Ca(NO₃)₂(s)+(NH₄)₂CO₃(l)→CaCO₃(s)+NH₄NO₃(l) Reaction 2

NH₄NO₃→N₂O+H₂O Reaction 3

49 g Ca(NO₃)₂ was added into 123 g CO₂-saturated 10 wt % ammonia aqueous solution. After stirring at 500 rpm for 5 minutes, the reaction was stopped. The solid was separated by filtration. The solid was calcium carbonate. The filtrate was carefully heated between 170-240° C., which decomposed into nitrous oxide and water vapor.

### Example C5

### Sequestration of CO₂ and Formation of Monoethanolammonium Nitrate

Reaction:

Ca(NO₃)₂(s)+CO₂−(OH—C₂H₄—NH₂)(l)→CaCO₃(s)+OH—C₂H₄−NH₃.NO₃(l)

16 g Ca(NO₃)₂ was added into 44 g CO₂-saturated 30 wt % MEA aqueous solution. After stirring at 500 rpm for 5 minutes, the reaction was stopped. The solution was separated by filtration. The filtrate was placed in an evaporating dish in an oven at 70° C. until it was dried. Monoethanolammonium nitrate was then formed. The product was stored in a desiccator as it was hygroscopic.

### Example C6

### Sequestration of CO₂ and Formation of Ethanamide

NH₃.H₂O+CO₂→(NH₄)₂CO₃ Reaction 1

(NH₄)₂CO₃+CH₃COOH→CH₃COONH₄+CO₂ Reaction 2

CH₃COONH₄→CH₃CONH₂+H₂O Reaction 3

30 g CO₂ was absorbed in 107 g 2M ammonia solution and 12 g acetic acid was added into the solution. CO₂ gas was then released and ammonium acetate solution was formed. The ammonium acetate solution was dehydrated under 95° C., producing about 15.4 g white ethanamide solid.

What is claimed:

1. A method of sequestering a multi-element gas comprising:
(i) providing a first solution comprising at least one gas absorber, the gas absorber comprises nitrogen and, in combination with a multi-element gas, is capable of forming an adduct;
(ii) contacting the first solution with the multi-element gas to promote the formation of the adduct, the adduct comprising the at least one gas absorber and the multi-element gas, which adduct is then present in solution;
(iii) providing a reactant comprising at least one element;
(iv) allowing the first solution comprising the adduct to contact the reactant under conditions that promote a reaction, wherein the multi-element gas in the adduct reacts with the reactant to form at least a first product and a second product in the solution, wherein the first product comprises at least one element of the multi-element gas, and wherein the second product comprises at least one element of the at least one gas absorber and at least one element of the reactant.

2. The method of claim 1, wherein the gas is emitted by an industrial plant.

3. The method of claim 1, further comprising heating the solution containing the second product to increase a concentration of the second product.

4. The method of claim 1, further comprising heating the solution containing the second product to form a third product comprising at least one element of the second product.

5. The method of claim 4, wherein the third product comprises a nitrogen-containing organic compound.

6. The method of claim 4, wherein the second product comprises a precursor to nitrous oxide.

7. The method of claim 1, wherein the gas is a greenhouse gas, a flue gas, or a combination thereof.

8. The method of claim 1, wherein the reactant is obtained from an industrial waste product.

9. The method of claim 1, wherein the first reactant comprises a halide element.

10. The method of claim 1, wherein the at least one gas absorber comprises ammonia, alkanolamines; polyamines of a mixed or single type; cyclic and aromatic amines; aminoacids; sterically free and hindered amines; monoethanolamine (MEA), diethanolamine (DEA), ethyldiethanolamine, methyldiethanolamine (MDEA), 2-amino-2-methyl-1-propanol (AMP), 3-piperidino-1.2-propanediol, 3-quinuclidinol, 2-piperidineethanol, 2-piperidinemethanol, N,N-dimethylethanolamine, 2-amino-2 methyl-1,3 propanediol, diisopropanolamine, piperazine, or combinations thereof.

11. The method of claim 1, wherein the first product comprises an inorganic compound comprising sulfide, sulfite, sulfate, carbonate, or combinations thereof.

12. The method of claim 1, wherein the second product comprises a nitrogen-containing organic species, halide, or combinations thereof.

13. The method of claim 1, wherein the reactant is a part of a solid which comprises at least one of (i) a material that can separate a gas molecule and (ii) a gas sequesteror.

14. The method of claim 1, wherein the gas comprises the element carbon, sulfur, oxygen, phosphorus, nitrogen, fluorine, or combinations thereof.

15. The method of claim 1, wherein the gas comprises carbon dioxide.

16. The method of claim 1, wherein the reactant comprises hydroxide ions.

17. The method of claim 1, wherein the reactant comprises an alkali metal element.

18. The method of claim 1, wherein the reactant is formed by reacting a reactant precursor with a basic solution.

19. The method of claim 18, wherein the basic solution comprises metal hydroxide.

20. The method of claim 1, wherein the reactant is obtained from a metal oxide, metal hydroxide, metal sulfate, metal fluoride, metal titanate, mineral silicate, mineral aluminosilicate, metal phosphate, or combinations thereof.

21. The method of claim 1, wherein step (iv) is carried out by a precipitation, ion addition, ion substitution, precipitation, disproportionation, or combinations thereof.

22. A ceramic article comprising a first product obtained from the method of claim 1.

23. A method of forming an organic compound from sequestering a multi-element gas comprising:
(i) providing a first solution comprising at least one gas absorber, the gas absorber comprises nitrogen and, in combination with a multi-element gas, is capable of forming an adduct;
(ii) contacting the first solution with the multi-element gas to promote the formation of the adduct, the adduct comprising the at least one gas absorber and the multi-element gas, which adduct is then present in solution;
(iii) providing a multi-element reactant;
(iv) allowing the first solution comprising the adduct to contact the reactant under conditions that promote a reaction, wherein the multi-element gas in the adduct reacts with the reactant to form at least a first product and a second product in the solution, wherein the first product comprises at least one element of the multi-element gas, and wherein the second product comprises at least one element of the at least one gas absorber and at least one element of the reactant; and
(v) heating the solution in the presence of a catalyst to form a third product comprising at least one element of the second product, wherein the third product comprises an organic compound.

24. The method of claim 23, wherein the catalyst comprises a metal halide.

25. The method of claim 23, wherein the catalyst comprises a halide salt of zinc, iron, aluminum, magnesium, or mixtures thereof.

26. The method of claim 23, wherein the catalyst is a dehydration catalyst.

27. The method of claim 23, wherein step (v) is carried out at between about 220° C. and about 250° C.

28. The method of claim 23, wherein the multi-element gas comprises hydrogen, carbon, sulfur, oxygen, phosphorus, nitrogen, fluorine, or combinations thereof.

29. The method of claim 23, wherein the gas comprises carbon dioxide.

30. The method of claim 23, wherein the third product is suitable for use in a pharmaceutical composition.

31. The method of claim 23, wherein the third product comprises piperazine.

32. The method of claim 23, wherein the at least one gas absorber comprising nitrogen is an amine.

33. A ceramic produced by a greenhouse gas or flue gas sequestering process, which process comprises reacting at least one component of a porous matrix with an adduct comprising a greenhouse or a flue gas and at least one gas absorber comprising an amine, which adduct is carried by an infiltrating medium to contact the at least one component of the porous matrix to provide at least a first product, thereby producing a ceramic.

34. The ceramic of claim 33, in which a remainder of the porous matrix acts as a scaffold for facilitating the formation of the first product.

35. The ceramic of claim 33, wherein the produced ceramic comprises a homogeneous microstructure.

36. The ceramic of claim 33, wherein the produced ceramic has a porosity of less than about 15%.

37. The ceramic of claim 33, wherein the produced ceramic has a porosity of less than about 5%.

38. The ceramic of claim 33, wherein the produced ceramic comprises a monolithic body.

39. The ceramic of claim 33, wherein the produced ceramic comprises particles physically bonded by ion substitution, ion addition, Ostwald ripening, or a combination thereof.

40. The ceramic of claim 33, wherein the produced ceramic is substantially free of hydraulic bonds.

41. The ceramic of claim 33, wherein the produced ceramic comprises crystalline inorganic materials, amorphous inorganic materials, or a combination thereof.

42. A nitrogen-containing compound produced by a gas separating or gas sequestering process, or a combination thereof, which process comprises reacting at least one component of a solid matrix with at least a first reactant that comprises at least one greenhouse or flue gas and which is present in a nitrogen containing infiltrating medium to provide at least a first product and a second product in the solution, wherein the first product comprises at least one element of the at least one greenhouse or flue gas, and wherein the second product comprises at least one element of the solid matrix and at least one element of the reactant; and heating the second product in the presence of a catalyst to form a third product comprising at least one element of the second product, whereby the third product produced comprises a nitrogen-containing compound.

43. The nitrogen-containing compound of claim 42, wherein the catalyst comprises a halide salt of a metal.

44. The nitrogen-containing compound of claim 42, wherein the compound comprises piperazine, a precursor to nitrous oxide, or monoethanolammonium nitrate.

45. The nitrogen-containing compound of claim 42, wherein the compound is suitable for use in a pharmaceutical composition.
